(19) United States
(12) Patent Application Publication
(10) Pub. No.: US 2013/0280307 A1
(43) Pub. Date: Oct. 24, 2013

(54) ENCAPSULATION OF MATTER IN POLYMER STRUCTURES

(71) Applicants: Matthew J. Fullana, Windsor, CT (US); Gary E. Wnek, Cleveland, OH (US)

(72) Inventors: Matthew J. Fullana, Windsor, CT (US); Gary E. Wnek, Cleveland, OH (US)

(21) Appl. No.: 13/866,106

(22) Filed: Apr. 19, 2013

Related U.S. Application Data
(60) Provisional application No. 61/635,859, filed on Apr. 19, 2012.

(51) Int. Cl. A61K 9/00

(52) U.S. Cl. CPC A61K 9/00
USPC 424/400

(57) ABSTRACT

A benign solvent for dissolving proteins and an agent comprises alcohol, salt and water. The protein is extracted from the solvent to form fibers or agglomerates that include the agent. Methods of extracting the protein include electrospinning the protein solution, electrospraying the protein solution, and a gravitational feed method to extract protein from the protein solution.

### REFERENCE TO PRIOR APPLICATION

This application claims the benefit and priority of U.S. Provisional Patent Application Ser. No. 61/635,859 filed on Apr. 19, 2012, and entitled “Encapsulation of Matter in Polymer Structures,” which is hereby incorporated herein by reference in its entirely.

### TECHNICAL FIELD

The disclosed material relates generally to forming polymer structures and more particularly to forming polymer structures that encapsulate other matter.

### BACKGROUND

Products and devices can be implanted into or applied onto a human body to treat injuries, diseases, and other conditions of the human body. The materials chosen for such products or devices can be important for the product or device to successfully treat conditions of the human body. For instance, the compatibility of a material with the human body can determine if the product or device can be positioned on or in the human body. Products and devices constructed from naturally occurring polymer materials such as proteins can provide biocompatible products or devices for implantation into or applying onto the human body to treat conditions of the human body.

### SUMMARY

A benign solvent for dissolving proteins comprises alcohol, salt and water. The ratio by volume of water to alcohol is between ninety-nine-to-one and one-to-ninety-nine. A salt concentration is between near zero moles per liter and the maximum salt concentration soluble in water. The amount of protein by weight as compared to the mixture of water and alcohol is between near zero percent and about 25 percent.

A method for forming a protein structure from a benign solvent comprises forming a benign solvent from water, alcohol, and salt; and dissolving a protein and an agent in the benign solvent to form a protein solution. The method further comprises extracting the protein from the protein solution; and arranging the protein into a protein structure such that the agent is encapsulated in the structure.

The method for forming a protein structure from a benign solvent further comprises electrospinning the protein solution to extract protein and the agent from the protein solution.

The method for forming a protein structure from a benign solvent further comprises electrospraying the protein solution to extract protein and the agent from the protein solution.

The method for forming a protein structure from a benign solvent further comprises using a gravitational feed method to extract protein and the agent from the protein solution.

### BRIEF DESCRIPTION OF THE DRAWINGS

Objects and advantages together with the operation of the invention may be better understood by reference to the detailed description taken in connection with the following illustrations, wherein:

FIG. 1 is a schematic illustration of apparatus for electrospinning protein fibers from a protein solution;

FIG. 2 is a schematic illustration of a Taylor cone;

FIG. 3A is a photograph of a pearl-on-a-string morphology;

FIG. 3B is a photograph of a pearl-on-a-string morphology;

FIG. 4A is a photograph of an embedded pocket morphology;

FIG. 4B is a photograph of an embedded pocket morphology;

FIG. 5A is a photograph electrospun fibers;

FIG. 5B is a photograph electrospun fibers;

FIG. 5C is a photograph electrospun fibers;

FIG. 5D is a photograph electrospun fibers;

FIG. 5E is a photograph electrospun fibers;

FIG. 5F is a photograph electrospun fibers;

FIG. 6 is a scanning electron microscopy image of electrospun fibers with pockets;

FIG. 7 is a scanning electron microscopy image of electrospun fibers illustrating crosslinking of the fibers;

FIG. 8 is a schematic illustration of a human eye;

FIG. 9 is a depiction of a thrombus forming on an implantable material;

FIG. 10 are photographs depicting the efficacy of nitric oxide on preventing the formation of thrombus on an implantable material;

FIG. 11 is a schematic representation of NOS domains, two paired enzyme units, the channeling of an electron with the participation of co-factors, and two PDB structures of NOS;

FIG. 12 is a schematic illustration of apparatus for electrospinning protein fibers from a protein solution;

FIG. 13 is a photograph of fibers resulting from electrospinning;

FIG. 14 is a photograph of fibers resulting from electrospinning;

FIG. 15 is a photograph of fibers resulting from electrospinning;

FIG. 16 a schematic representation of an embedded aqueous node inside fiber matrix;

FIG. 17 is a chart comparing a layer-by-layer method of deposition of NOS on the surface of an implantable biomaterial with an electrospun method;

FIG. 18 is a chart depicting the electrochemical characterization of embedded fibers; and

FIG. 19 FIG. is a schematic illustration of apparatus for forming of protein structures from a protein solution.

### DETAILED DESCRIPTION

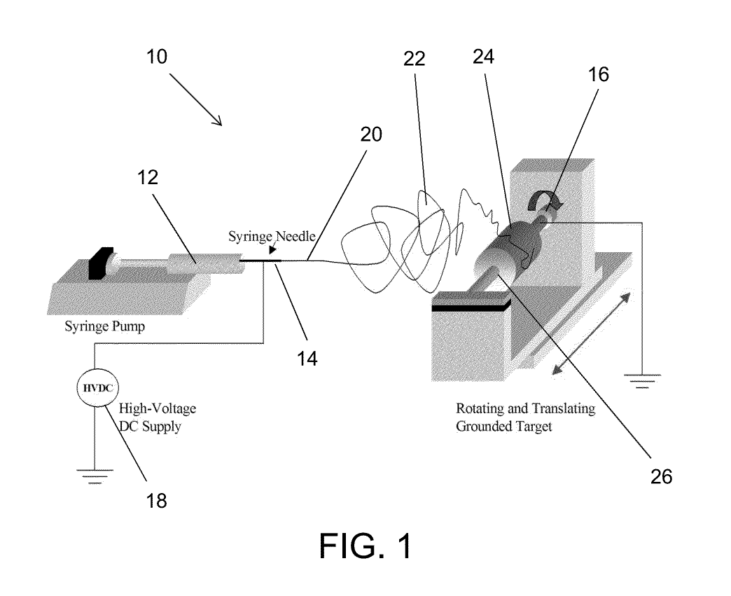

The apparatuses and methods disclosed in this document are described in detail by way of examples and with reference to the figures. It will be appreciated that modifications to disclosed and described examples, arrangements, configurations, components, elements, apparatuses, methods, materials, etc. can be made and may be desired for a specific application. In this disclosure, any identification of specific shapes, materials, techniques, arrangements, etc. are either related to a specific example presented or are merely a general description of such a shape, material, technique, arrangement, etc. Identifications of specific details or examples are not intended to be and should not be construed as mandatory or limiting unless specifically designated as such. Selected examples of apparatuses and methods for forming biocompatible protein structures with encapsulated agents from a solution of protein and the agent dissolved in a benign solvent are hereinafter disclosed and described in detail with reference made to FIGS. 1-19.

Naturally occurring materials are good candidates for products and devices that are intended for use with biological material such as human and animal tissue. One category of materials that can be compatible with biological material is natural polymers such as proteins. Examples of such biocompatible proteins include, but are not limited to, collagens, gelatin, elastin, fibrinogen, silk, and other suitable proteins. Such proteins can be used to form protein structures for implantation into or application onto a human body. Other materials that are generally biocompatible are polysaccharides such as hyaluronic acid, chitosan, and derivatives of starch and cellulose such as hydroxypropyl methyl cellulose phthalate, deoxyribonucleic acid (DNA), and ribonucleic acid (RNA).

One example of a protein structure that can be useful in forming products and devices for the human body is protein fibers. Protein fibers can be used to form a scaffold or porous mat. Such scaffold or porous mat structure can mimics an extra cellular matrix (ECM) of human tissue. Natural ECM generally has an open and porous structure. As will be described herein, fibers formed from proteins and joined into a matrix can simulate such an open and porous structure. Such a protein structure can be used in tissue engineering or wound care as a substrate for growing cells and/or tissue.

In another example, protein fibers can be formed such that an active agent is integrated into the protein fibers. Such agents include pharmaceutical agents, drugs, chemicals, compounds, and any other such agents that can have an effect on biological material. Agents can be integrated into fibers in a number or methods. For example, a fiber can include one or more pockets or voids that can accommodate an agent and encapsulate the agent. In another example, an agent can penetrate, permeate, or be integral to the fiber such that the agent is embedded in the fiber. The agent can be encapsulated or embedded in the fiber such that the agent can be released, discharged, or leached from the fiber over time. It will be understood that such arrangement can provide for the fibers and/or scaffolds to be systems for delivering the agent to biological material that come into contact with or is proximate to the fibers.

In another example, proteins can be used to form structures such as, for example, generally spherical agglomerates. Such agglomerates can be formed in a variety of sizes, ranging from submicron diameters to several hundred micrometers in diameter. Because of the compatibility of proteins with human tissue, protein agglomerates can be successfully implanted in or passed through the human body to affect treatment of a medical condition. As described for fibers, the agglomerates can be embedded with, infused with, or encapsulate agents such as pharmaceutical agent, drugs, chemicals, compounds, and any other such agents that can have an effect on biological material. Similarly, protein agglomerates can function as a component of systems for delivering agents to biological material. As will be understood, a drug or other useful chemical compound can be attached to or inserted into a protein agglomerate. The protein agglomerate can then be passed through the human body, including through the blood stream, to a desired location where the drug or other useful agent can be released either at once or over time. In another example, protein agglomerates can function as structural or supportive components in the human body. For instance, protein agglomerates can be used in cosmetic medicine. Protein agglomerates can be injected under the skin to support the skin and smooth out wrinkles.

Biocompatible polymer materials can be used in the human body to restore and improve physiologic function and enhance survival and quality of life with minimal cytotoxic effects. For example, polymeric scaffold structures can be arranged for placement onto or into a human eye and/or adapted for other uses. For example, a polymeric scaffold or other structure can be adapted for delivery of hydrophobic drugs to biological material such as tissue. For example, in one embodiment, protein fibers can incorporate and release hydrophobic drugs, such as dexamethasone, from a hydrophilic polymeric matrix produced by electrospinning Naturally-derived polymers, such as gelatin and collagen, can be electrospun under various conditions to generate different fiber morphologies. In addition, synthetic hydrophilic polymers, such as polyvinyl alcohol were also electrospun to form useful morphologies. Collagen scaffolds are an example of a protein useful for biomedical devices given the high concentration of collagen present within tissue. Gelatin, a denatured form of collagen, is another example that can be utilized. Techniques such as fluorescent microscopy, SEM, and UV/Visible spectrometry can be used to characterize electrospun fiber diameter, structure, drug incorporation and kinetic release profile.

One method of forming a protein structure begins with dissolving a protein such as collagen or gelatin in a solvent. Once dissolved, the protein can be extracted from the solvent and organized into a protein structure. Generally, a benign solvent is a solvent that reduces health risks to a human body or is of minimal risk to the health of a human body.

One example of a benign solvent for dissolving protein comprises water, alcohol, and salt. The protein can be a collagen or gelatin or any other naturally occurring or synthetic polymer. The alcohol can be ethanol, and the salt can be sodium chloride (NaCl). The association between water molecules, salt, and alcohol creates a complex structure in which proteins such as collagen and gelatin are substantially soluble. Collagen and gelatin are substantially soluble in suitable water-alcohol-salt benign solvents because the properties of the solvents screen interpeptide interaction that usually results in insolubility of such naturally occurring polymers. For example, the electrostatic interaction between the salt and the carbonyl group of the hydrophilic part of collagen or gelatin and the hydrophobic interaction between the hydrocarbon chain of ethanol and the hydrophobic part of collagen or gelatin can screen such interpeptide interaction. In general, any molecule or complex that exhibits a hydrophilic part and a hydrophobic part spaced by approximately the same distance as the hydrophilic part and hydrophobic part of the collagen or gelatin molecule can dissolve collagen or gelatin.

Generally, in suitable water-alcohol-salt solvents, the ratio of water to alcohol can range from a volume ratio of about 99:1 to about 1:99, the salt concentration can range from near 0 moles per liter (M) to the maximum salt concentration soluble in water, and the amount of protein by weight (as compared to the solvent) can range from near 0 percent to about 25 percent. In one example, the benign solvent comprises about a one-to-one ratio of water to ethanol and a salt concentration of about 3 M NaCl. In one example, collagen is dissolved in such a solvent until the solution reaches about 16 percent collagen by weight. In another example, the solution comprises semed S (principally collagen type I with a ca. 5 percent collagen type III) dissolved in a solvent comprising phosphate buffered saline (PBS) buffer and ethanol, where the buffer to ethanol ratio of about one-to-one by volume. The saline concentration in the PBS buffer can range from 5× to 20×. The collagen concentration can be for example about 16 percent as compared to the total weight of the PBS/ethanol solvent. In yet another example, the protein dissolved in the solvent can be gelatin. The solvent can comprise a PBS buffer with a salt concentration of 10× mixed with ethanol at a one-to-one ratio by volume. Gelatin can be dissolved until the amount of gelatin by weight is about 16 percent by weight.

When protein has been dissolved in a suitable water-alcohol-salt solvent to form a protein solution, suitable processing methods can be used to extract protein from the solution and form protein structures. As previously discussed, such protein structures can be implanted into or applied onto the human body to affect treatment of a condition. Examples of suitable processing methods include, but are not limited to, electrospinning, electrospraying, and gravitational feed methods.

In one example, electrospinning can be used to form a protein structure. An example of apparatus 10 for forming a protein structure by electrospinning protein dissolved in a benign water-alcohol-salt solution is schematically shown in FIG. 1. The electrospinning method can include placing the protein solution in a syringe 12. The syringe can include a metal needle 14. The protein solution in the syringe 12 can be charged by the application of an electrical potential between the metal needle 14 and a ground target 16 spaced a distance away from the metal needle 14. The electrical potential can be applied by charging the metal needle 14 with a voltage from a power supply 18. The electrical potential can be increased until the electrostatic forces in the protein solution overcome the surface tension at the tip of the metal needle 14. As this surface tension is overcome, a fine jet 20 of solution containing entangled protein chains can be drawn out of the metal needle 14. As the fine jet 20 travels through the air, at least a portion of the solvent evaporates, resulting in a protein fiber 22 that dries as it travels through the air. The dry protein fiber 22 can be collected on a surface 24 that is in contact with the ground target 16. As shown in FIG. 1, the surface 24 can be on a rotating cylinder 26. The surface 24 can also be arranged to be static or designed to oscillate in rotational or translational directions. It will be understood that the electrical potential can be created using a direct current (DC) power supply or an alternating current (AC) power supply.

The architectural structure of protein mats or scaffolds can be important depending on the intended application of the mat or scaffold. For example, mats and scaffolds can be used to simulate types of human tissue or be arranged to deliver and release pharmaceutical or other agents in the human body. Aligned fibers may be useful in simulating a variety of tissue types including ligaments, nerves, cardiac tissues, and the like. The alignment of electrospun fibers may be controlled by the rotational speed of the rotating cylinder 26 shown in FIG. 1. If the speed of the cylinder matches or is faster than the speed of the jet of protein solution exiting the syringe, the protein fibers may be drawn out of the syringe in the loop direction of the cylinder. The orientation of protein fibers in the mat can be characterized by Herman's orientation function, which is:

f=(3*(cos² θ)−1)/2

where θ is the angle of the protein fibers compared to the loop direction of the drum.

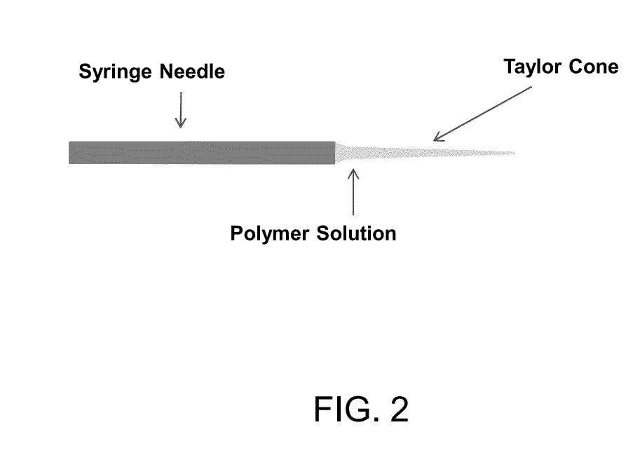

An optimally aligned fiber mat (that is to say, a mat where all the fibers all aligned in the same direction) will have a Herman's orientation function equal to 1. An optimally random configured fiber mat (that is to say, a mat where all the fibers are randomly aligned) will have a Herman's orientation function equal to −0.5. In addition, electrospinning involves the formation of the Taylor cone, illustrated in FIG. 2. When the polymer solution overcomes the surface tension of the solvent and yields fibers in the presence of the electric field a Taylor cone is formed. An inconsistent Taylor cone may result in spraying, or inconsistent fiber diameters, or even failure to form a fiber, all of which will impact the mechanical properties and performance of the resulting polymer structure.

The arrangement illustrated in FIG. 1, includes a number of variables, each of which can be optimized based on the polymers in use and the final desired properties of the scaffold and its fibrous mesh. For example: 1) properties of the polymer such as molecular weight (Mₙ and M_{w}), molecular weight distribution, and polymer structure; 2) properties of the solution such as solvent, vapor pressure, solution viscosity, surface tension, solution conductivity, polymer concentration, and effective T_{g}; 3) process parameters such as distance from the Taylor cone to collection site, rotational and translational speed of the target, applied voltage, flow rate, and spin duration; and 4) environmental parameters such as temperature, humidity, and air circulation can all be considered in arranging a process for optimizing the final properties of the polymer structure.

In one example, the protein solution can be placed in a 5 milliliter (ml) syringe equipped with a 21 gauge blunt needle. The syringe can be placed in a syringe pump. A rotating drum can be placed approximately 10 centimeters (cm) from the tip of the needle. The pump rate can be set to about 1 milliliter per hour (ml/h) and the electrical potential can be set to about 20 kilovolts (kV). The result of such a setup can include the formation of a scaffold or mat on the rotating drum containing randomly oriented fibers or quasi-aligned fibers. The electrospinning process parameters, such as flow rate, potential field, and needle-to-collector distance can be adjusted to produce a variety of results or to optimize the stability of the fine jet of solution during electrospinning.

In another example, a scaffold or mat can be electrospun from a solution of about 16 percent by weight of gelatin or collagen dissolved in a PBS (10×) and ethanol solution with a volume ratio of about one-to-one. A flow rate of about 1 ml/h, a potential field of about 20 kV, and a needle-to-collector distance of about 10 cm can produce a stable jet of gelatin drawn from the gelatin solution.

As previously noted, non-woven, electrospun porous scaffolds can mimic the extracellular matrix. Such structures can be useful in delivery of agents such as pharmaceutical agents and drug and in, for example, cell seeding. Electrospinning proteins such as collagen and gelatin can result in the spinning of fibers as shown in FIG. 1. Such fibers can be highly aligned or oriented when mats and scaffolds are formed. In one example, electrospinning may be used to draw out protein fibers and such fibers can be generally arranged in a matrix. Once the fibers are arranged in a matrix, the fibers can be cross-linked to mimic the structure of ECM. Once cross-linked, the formed mat or scaffold can be a non-water-soluble protein structure that is biocompatible with the human body and thus implantable into or applicable onto the human body.

As the mat or scaffold is being formed by electrospinning, the fibers can be arranged so that fibers overlay one another and are in contact with one another. While in such an arrangement, the physical structure of the mat or scaffold can be enhanced by cross-linking the protein fibers. In one example, end groups such as aldehyde, carbodiimide, or epoxy can facilitate the cross-linking of the protein fibers of the mat or scaffold. A carbodiimide such as 1-ethyl-3-(3-dimethylaminopropyl)carbodiimide hydrochloride (EDC) can cross-link collagen using N-hydroxysuccinimide (NHS) as a catalyst. An electrospun collagen mat can be immersed in a 200 mM EDC and NHS ethanol solution for approximately 4 hours to cross-link collagen fibers. Once cross-linked, the collagen mat or scaffold can be placed in a PBS and salt solution similar to the buffer described above. Such a step can remove any non-cross-linked collagen from the mat or scaffold. The mat and scaffold, which can mimic the extra cellular matrix of human tissue can now be used as a substrate to grow cells or tissue, or can be used as a covering for an open wound to promote growth of tissue of the wound.

Examples of methods for forming a protein mat or scaffold by electrospinning can include adjusting the protein's solubility in the benign solvent; adjusting the evaporation rate of the solvent; adjusting the viscosity of the solution; or adjusting the surface tension of the solution. In one example, the solubility of collagen is enhanced by the addition of a salt to a water and ethanol mixture with a generally neutral pH level. When about 5 percent by weight of NaCl is added to a water and ethanol mixture for an about 16 percent by weight collagen solution, substantially all collagen dissolves. In another example, the salt composition of a PBS buffer solution can be about 80 percent NaCl by weight, about 17.4 percent sodium phosphate anhydrate by weight, and about 2.4 percent potassium phosphate anhydrate by weight. In yet another example, collagen may be dissolved in a PBS buffer where the total salt concentration exceeds 5 percent by weight. The evaporation rate of the solvent can be increased by increasing the amount of alcohol as compared to water in the protein solution.

As will be understood, the pH level, temperature, type of collagen, and type and concentration of salt all influence the structure of collagen in the protein solution. For example, at low collagen concentrations and a pH level of about 7.4, the transition temperature of crystalline polymer to random coil polymer is about 45 degrees Celsius. The transition temperature can be independent of salt concentration for potassium chloride (KCl) and NaCl. There is a progressive decrease in precipitation of collagen, that is to say that collagen becomes more soluble, as more salt is added. Addition of salt results in destabilization of the precipitated collagen while the ionic strength increases with salt additions. Collagen solubility can increase even if it appears that the crystalline structure of collagen is maintained upon addition of salt.

Alcohol affects the solubility of collagen in the buffer and ethanol solution. Alcohol and collagen interaction is moderated by hydrocarbon chain length, with alcohol disrupting internal hydrophobic interactions in the collagen. With increased alcohol concentration, there is a progressive increase in molar destabilization of the crystalline collagen precipitated in an alcohol and potassium acetate buffer mixture at an acidic pH, for example, a pH of about 4.8. For single collagen molecules, structural stability is primarily a function of interpeptide hydrogen bonding and chain rigidity.

The addition of salt promotes the solubility of collagens. Hydrogen bonding between the hydrophilic part of collagen and water molecules can be too weak to break the interpeptide interaction, and the stronger electrostatic forces induced by salt in aqueous media may be necessary. The combination of both electrostatic and hydrophobic forces appears to interact strongly enough with the collagen chain to substantially dissolve the collagen in a mixture of ethanol and PBS buffer with an about one-to-one ratio when a salt concentration is at least about 5× in the buffer.

In addition to dissolving proteins such as collagens, the buffer and ethanol binary solvent can further facilitate the electrospinning process. The salt in the buffer as well as the alcohol can assists in overcoming the high surface tension of water that can partially inhibit spinnability of water based polymeric solution. In addition, the salt increases the charge density in the protein solution, which can facilitate the formation of a stable Taylor cone. The low evaporation rate of water, which can inhibit the formation of fibers during electrospinning, can be compensated for by the high evaporation rate of alcohol.

Electrospinning of collagen solutions with about a one-to-one volume ratio of ethanol to PBS (10× or 20×) can be stable and create fiber mats or scaffolds that exhibit relatively consistent fiber diameters. The increase of salt concentration in the PBS buffer can decrease the fiber diameter, and higher salt concentration can result in greater elongation of the electrospun jet due to higher density of repulsive charges in the Taylor cone. Increasing the salt concentration from 10× to 20× can decrease the average fiber diameter but also can significantly reduce the standard deviation of the fiber diameter distribution.

As will be understood, cross-linking of protein mats or scaffolds facilitates the use of electrospun mats or scaffolds for regenerative or tissue engineering and wound care, because cross-linking promotes stability of the collagen mat or scaffold. In addition to mimicking the ECM of human tissue to promote cell or tissue growth, when collagens with hemostatic properties are used, application of a mat or scaffold over a new or existing wound can arrest blood flow from the wound and promote clotting. Furthermore, it will be understood that including an agent such as an antibiotic drug with the fibers of the scaffolds can further assist in wound care and regeneration of tissue.

Cross-linking can be facilitated by the presence of carboxyl groups on the hydrophilic part of natural polymers such as collagens a gelatins. Cross-linking of fiber mats can be achieved with EDC and NHS as a catalyst. Mats can be immersed in an ethanol solution comprising EDC and NHS for a period of time, and then mats can be immersed in a buffer solution containing the same salt concentration as the one the collagen was electrospun from to remove un-cross-linked fibers.

A collagen mat can be soaked in an ethanol solution such that the mat shrinks to form a film-like surface. If shrinkage is not desired, frames can be placed on each side of the mat and clipped together to prevent the mat from shrinking when it is immersed in the ethanol solution. Such frames can be constructed of material that is easy to remove, such as Teflon. The fiber diameter distribution does not significantly change between non-cross-linked and cross-linked collagen when a frame is used. Therefore, the frame can efficiently prevent fiber shrinkage when immersed in ethanol.

As previously discussed, gelatin may be electrospun from binary solutions and electrospinning conditions disclosed herein. For example, when a PBS (10×) and ethanol solution is used for dissolving gelatin at about 16 percent by weight, similar results are obtained as compared to collagen fibers. In addition, the gelatin can be cross-linked in a similar manner and under similar conditions as described for collagens.

The concentration of collagen in the solution may affect electro spinning. Collagens readily dissolve in a solvent comprising a one-to-one ratio between PBS (20×) and ethanol. Generally, collagen solutions ranging from about 4 percent by weight to about 25 percent by weight can be electrospun. By controlling the concentration of collagen, different morphologies and fiber diameters can result. Generally the diameter of the fibers can be inconsistent because the viscosity of the solution is low and does not generally form continuous fibers during electrospinning. As the concentration of collagen is increased, the diameter of the fibers becomes more consistent because continuous fibers are more readily generated by a solution with about 10 percent collagen by weight.

The concentration of salt and ethanol can affect the solubility of collagens in water. Collagen can be generally insoluble at about 16 percent by weight in either PBS (20×) or ethanol. However, when a small amount of ethanol is added into PBS (20×) buffer to form a PBS (20×) to ethanol volume ratio of about nine-to-one, the collagen substantially dissolves into this mixture. By adding more ethanol into PBS (20×) buffer (that is, the volume ratio decreases from about nine-to-one to about seven-to-three to about one-to-one) there is generally no affect on the solubility of collagen. The collagen remains substantially soluble. However, when the PBS (20×) to ethanol volume ratio is reduced to three-to-seven, collagen is generally no longer soluble. Furthermore, the salt concentration affects the solubility of collagen when the water to ethanol volume ratio is held constant at about one-to-one. The salt concentration in 5×, 10× and 20× PBS buffer is sufficient to substantially dissolve collagen in the mixture solution.

The addition of salt and ethanol to the protein solution can facilitate the electrospinning of the polymer solution. As salt increases the conductivity and ethanol decreases the boiling point, concentrations of salt and ethanol affect the electrospinnability of solutions that are capable of dissolving collagen with PBS (20×) to ethanol ratio varying from about nine-to-one to about one-to-one. Fibers may be formed from electrospinning collagen with PBS (20×) to ethanol volume ratios of about seven-to-three. In addition, collagen solutions with a PBS (20×) to ethanol volume ratio of about one-to-one demonstrate good electrospinability and a stable Taylor cone. Such a solution may be electrospun to form fibers and a mat as thick as about 150 microns.

In one example, the protein solution can include a cross-linking agent so that cross-linking of protein fibers occurs as the protein fibers are being electrospun. This reduces the formation and cross-linking of protein fibers to one general step. In such an example, the protein solution includes protein, water, alcohol, salt, and a cross-linking agent. A protein solution is formed by dissolving about 16 percent by weight of collagen in a solvent. The solvent comprises PBS buffer (20×) and alcohol. Prior to forming the solvent a cross-linker is added to the alcohol. The cross-linker can be about 200 mMoles of EDC and NHS at a ratio by weight of about one-to-one. The collagen solution can be deposited in a syringe equipped with a metal needle as previously described. The protein solution is subjected to an electrical potential and electrospun to form a jet of protein solution and form a protein structure such as a mat or scaffold. In one example, a voltage of about 20 KV can be applied to the metal needle and the pump rate can be about 0.5 milliliters per hour. A rotating drum can be positioned about 10 centimeters from the needle to collect the electrospun mat.

As previously noted, protein fiber scaffolds and other protein structures can be used as a delivery systems for pharmaceutical and other suitable agents for delivering such agents to areas of the human body. For example, a therapeutically-active small molecules and/or macromolecules can be incorporated, embedded, or encapsulated into polymer matrices in useful forms (e.g., films, fibers, dispersions) in an effort to control the rate of delivery of such substances to biological material situated in contact with or proximate to the matrix. Nano-fiber or micro-fiber mats can provide versatility in terms of polymer choice and ease of fabrication. A wide range of polymers are capable of being processed into small-diameter fibers, and non-woven mats can be utilized for tissue engineering and regenerative medicine applications. Such mats can also include drug delivery functionality, releasing any number of small-molecule and/or macromolecular substances at a controlled rate. The process of electrospinning can be utilized to control fiber morphology, producing fibers having diameters ranging from 100-200 nanometers up to several microns. In one example, a hydrophobic drugs can be encapsulated, embedded or otherwise incorporated into or within hydrophilic polymer fibers formed by electrospinning One example of an electrospinning process to achieve encapsulation of an agent is a “two-phase” or suspension approach. A suspension of a hydrophobic polymer such as PLGA in chloroform and protein in buffer can be prepared by agitation and then electrospun to produce polymer fibers with small, aqueous pockets containing protein. Such a process can also be used to encapsulate hydrophobic pharmaceutical agents, such as dexamethasone, a hydrophobic anti-inflammatory agent, into hydrophilic polymers, such as collagen or gelatin. A pharmaceutical agent can be mixed with a protein solution as described herein and upon electrospinning fibers with a “pearl-on-a-string” configuration and an “embedded pocket” configuration can be formed. In such configurations, the pharmaceutical agents can be encapsulated or otherwise incorporated into the protein fibers.

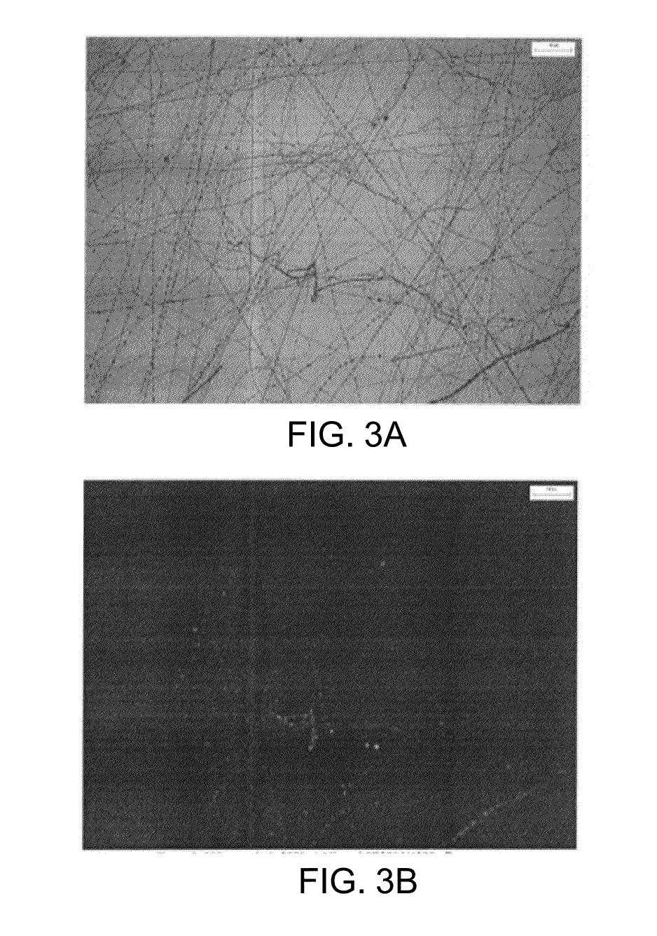

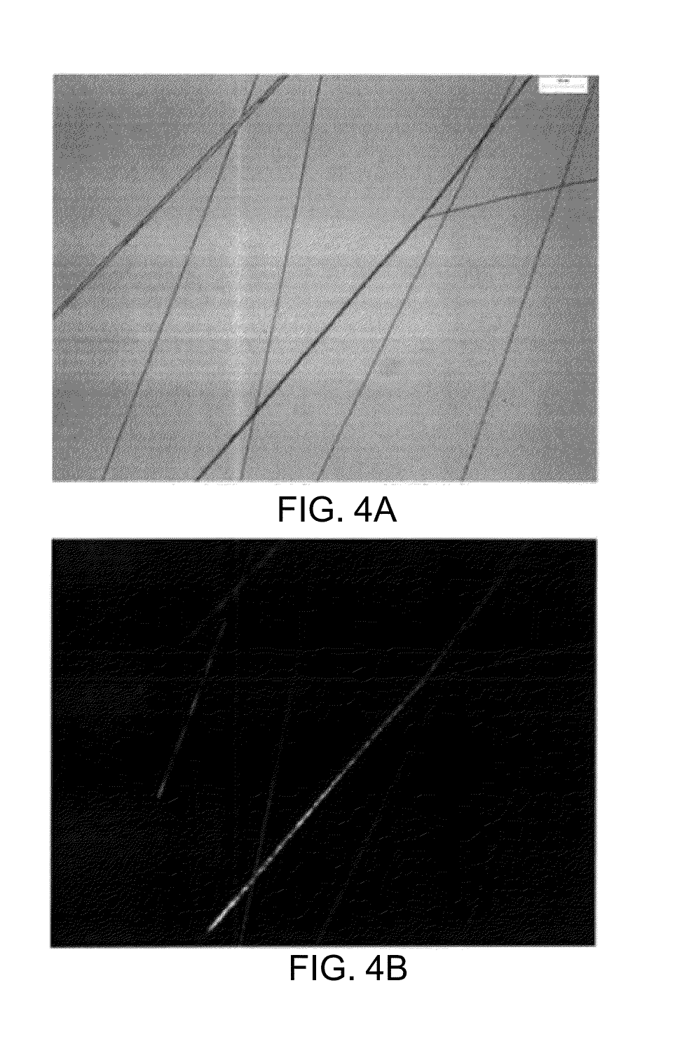

Representative photographs of a pearl-on-a-string morphology are shown in FIGS. 3A and 3B. Representative photographs of an embedded pocket morphology are shown in FIGS. 4A and 4B. A pharmaceutical agent can be mixed with collagen, or other suitable proteins in a solution. For example, bovine-derived gelatin of varying concentration can be dissolved in a benign solvent mixture of 1:1 ethanol/phosphate-buffered saline (20×). To demonstrate such a process, an oil (either vegetable or mineral) contained a fluorescent, hydrophobic dye can be used. The oil/dye solution can be mixed with the gelatin and electrospun to produce micron-sized fibers with the two different morphologies: a pearl-on-a-string configuration and an embedded pocket configuration where the oil and hydrophobic dye are encapsulated within the gelatin fibers. The specific parameters used for the fibers shown in FIGS. 3A, 3B, 4A, and 4B are: 200-300 mg of bovine-derived gelatin mixed with 1 mL of a 1:1 ratio of ethanol and PBS 20× buffer. The solution can be stirred until the gelatin was fully dissolved (approximately 10-15 min). A homogeneous solution containing 0.5 mg/mL of orange 2G hydrophobic dye/oil is prepared. Varying amounts of oil can be added to the gelatin solution, ranging from 50-350 μL. The solution is then sonicated for about 15 minutes to disperse the oil in the gelatin solution, forming a cloudy suspension. The mixture is loaded into a syringe with an 18-gauge, blunted needle and electrospun onto a rotating drum using a delivery rate of 0.5 ml/hr, with an air gap distance of 12 cm and an applied voltage of 21-30 kV.

When viewed under a light microscope at 20× magnification, the fibers spun from solutions with high oil concentrations (250 μL) and lower gelatin concentrations (0.2 g/mL) exhibited a pearl-on-a string configuration as shown in FIG. 3A. As shown in FIG. 3B, upon excitation at 450-490 nm these pearl-like beads fluoresce indicating that the beads are oil “pockets” containing the fluorescent dye.

Fibers spun from solutions containing low oil concentrations (125 μL) and a higher gelatin concentration (0.3g/mL) exhibited a different morphology than those with higher oil concentrations. When viewed in bright field the low oil concentration fibers do not appear to have any bead formation, as shown in FIG. 4A. However when excited at 450-490 nm the fibers fluoresce (as shown in FIG. 4B) indicating that there are oil domains embedded within the fibers as opposed to the pearl-on-a-string fibers seen at higher oil concentrations.

As shown by FIGS. 3A, 3B, 4A, and 4B and the accompanying description, gelatin fibers can be mixed with a pharmaceutical agent and electrospun from a solution of PBS and ethanol to encapsulated, embed, or otherwise incorporate the pharmaceutical agent. As will be understood, a pharmaceutical agent that is effective for wound healing, bacteria suppression, or any other medical applications can be encapsulated, embedded, or otherwise incorporated in protein fibers.

In another example, gelatin or collagen scaffolds that encapsulate a pharmaceutical agent (such as, for example, a hydrophilic dexamethasone) can be prepared by dissolving either material in a 1:1 ratio mixture of PBS 20× and ethanol and adding the pharmaceutical agent. EDC and NHS can be used for crosslinking The solution can be drawn into a syringe fitted with an 18 gauge blunted needle, with a voltage of 20-22 kV applied. The collection target can be placed at a working distance of 12 centimeters. The environment can be maintained at a relative humidity of about 20% using a nitrogen gas purge. In a demonstration of how the encapsulation works, a red florescent dye solution dissolved in vegetable oil can be used in lieu of the pharmaceutical agent dexamethasone. As described above, parameters can be adjusted to optimize electrospinning to yield optimum fibers.

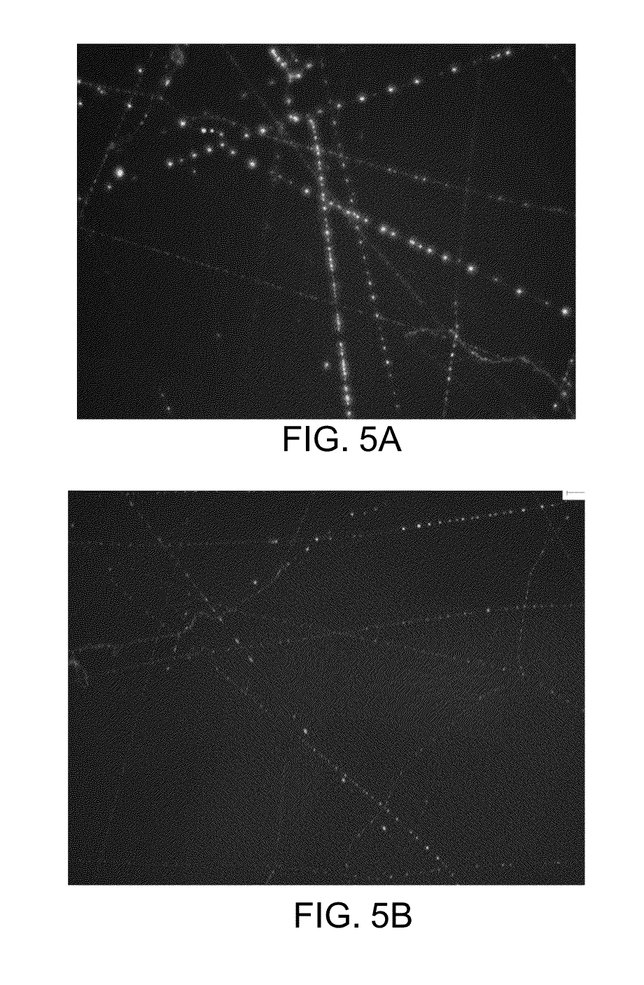

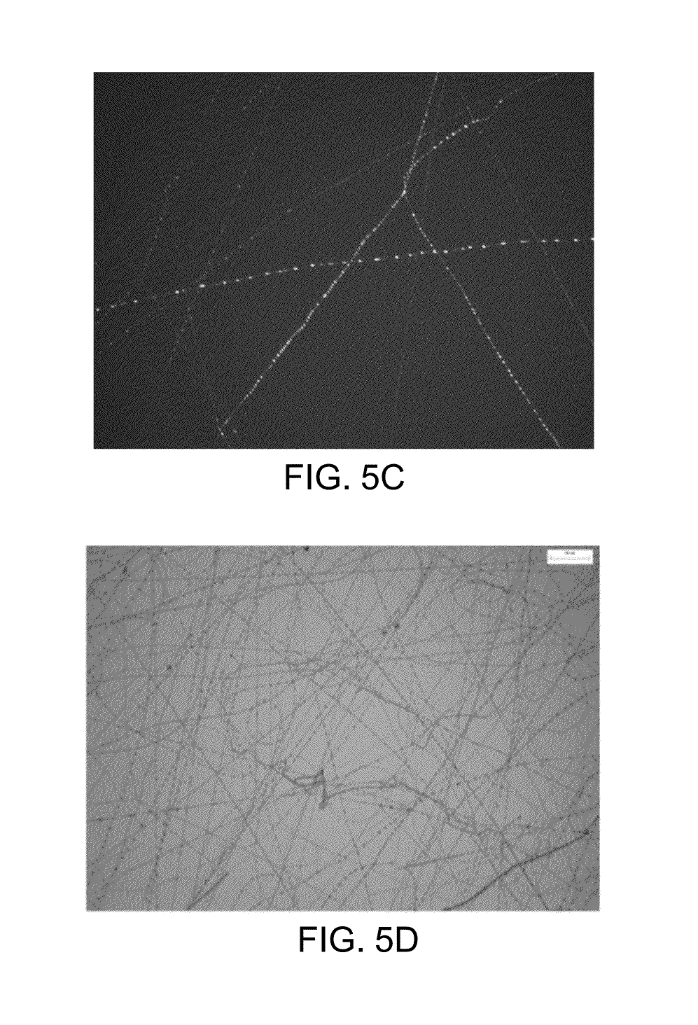

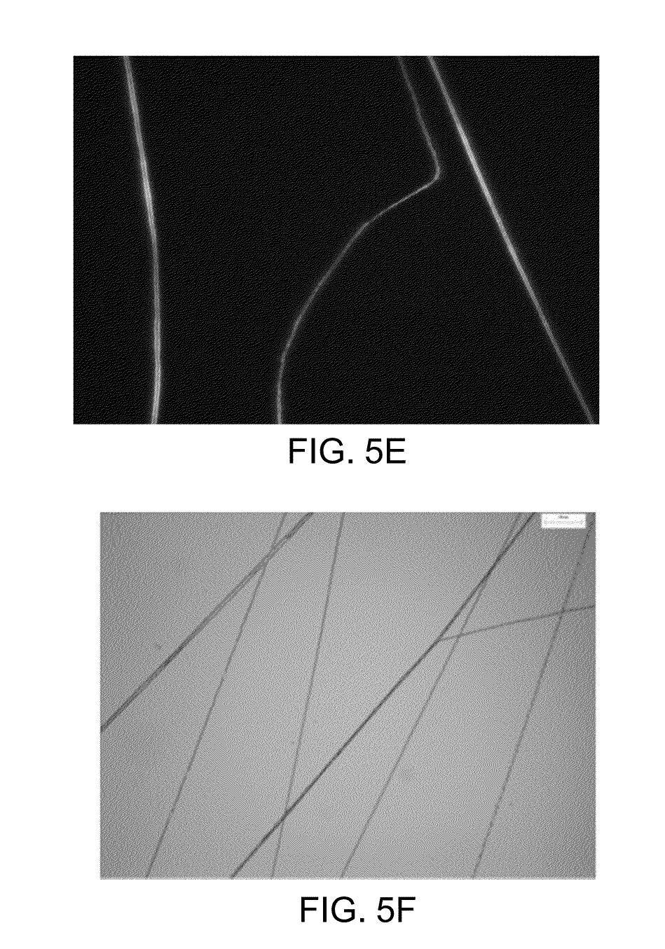

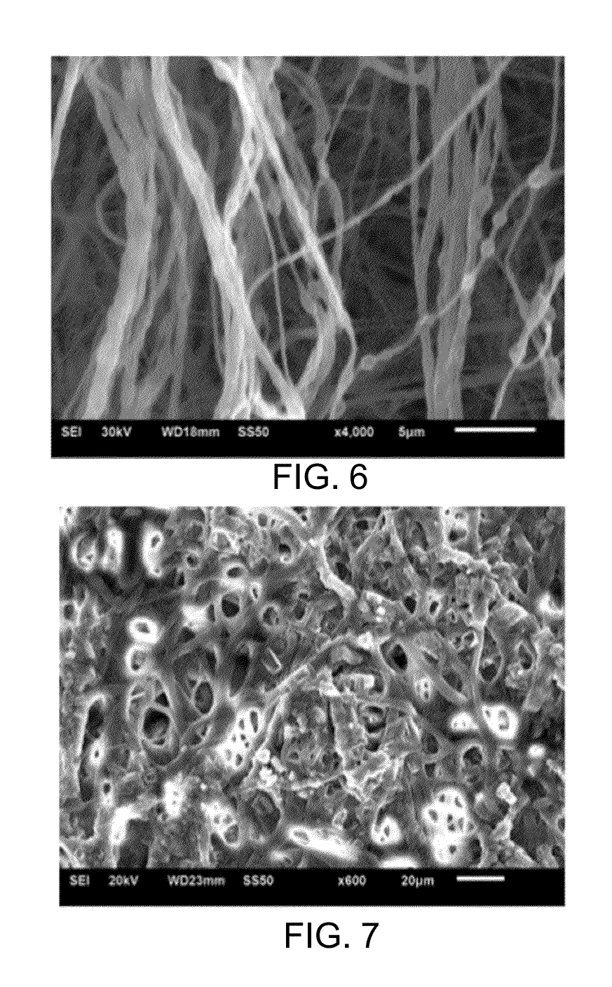

FIGS. 5A-5F show photographs of gelatin and polyvinyl alcohol (PVA) fibers electrospun with different concentrations of vegetable oil with dissolved red florescent dye added to the protein solution. FIG. 5A has a concentration of 350 μL of oil/dye. FIG. 5B has a concentration of 125 μL of oil/dye. FIG. 5C has a concentration of 125 μL of oil/dye. FIG. 5D has a concentration of 250 μL of oil/dye. FIG. 5E has a concentration of 150 μL of oil/dye. FIG. 5E has a concentration of 250 μL of oil/dye. FIG. 6 is a scanning electron microscopy image of electrospun fibers with “pockets.” FIG. 7 is a scanning electron microscopy image of electrospun fibers illustrating crosslinking of the fibers.

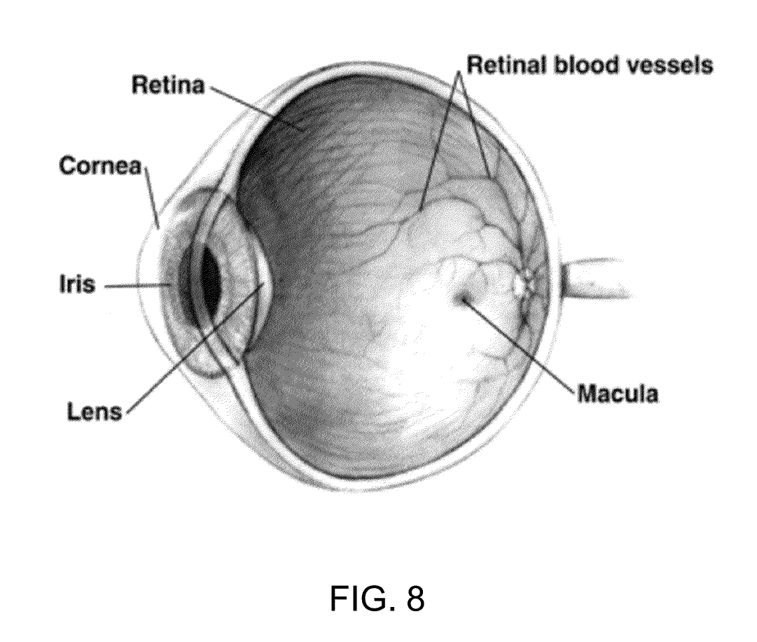

One application for the above described pharmaceutical agent encapsulated fibers is for treatment of the human eye. The human eye (shown in FIG. 8) is extremely hydrophilic. Application of a hydrophilic polymeric scaffold containing a hydrophobic pharmaceutical agent into or onto the eye can improve the absorption of the agent into the tissue resulting in better efficacy. Gelatin and collagen, among other proteins, can be used because of their biocompatibility and similarity to native tissues. In another example, PVA, in addition to being biocompatible, can impart more mechanical stability for an electrospun scaffolds. In one example, a patient suffering from macular degeneration, the macula, which is the central part of the retina containing the most clear vision, degrades. Patients can experience a loss of clear vision and the inability to fully see objects. Implantation of this scaffold incorporating dexamethasone or seeded with cells can facilitate regenerative growth and healing of the macula, consequently leading to better vision and avoiding painful injections into the eye and improving patient compliance and quality of life.

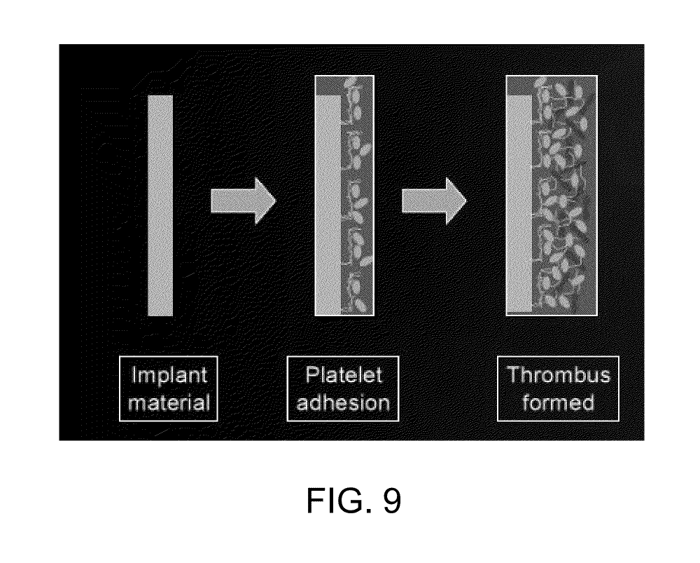

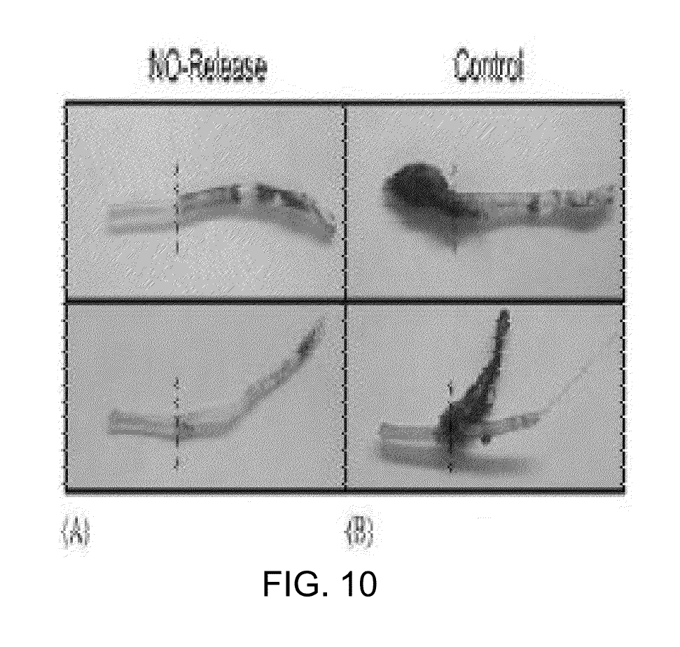

Another application for encapsulating agents in electrospun fibers is a treatment to prevent thrombosis at the surface of medical implant material and restenosis that can result from implantation of materials in the human body. As will be understood, thrombosis on the surface of foreign devices implanted or used as part of cardiovascular procedures or restenosis can be problematic for patients. Agents are known to counteract platelet aggregation in mammalian physiology. For example, nitric oxide is known to counteract such platelet aggregation. At measured release levels, nitric oxide can slow or prevent the thrombosis cascade on the surface of blood-contacting medical implants. Nitric oxide synthases (NOS)—enzymes responsible for catalytic conversion of the substrates L-arginine to nitric oxide and L-citrulline—can be used to fabricate nitric oxide releasing biomaterial in its closest native characteristics to mammalian tissue. Such modification can address the issue of thrombosis on the surface of foreign devices implanted in such procedures as cardiovascular procedures and restenosis. FIG. 9 schematically illustrates thrombosis forming at the surface of a medical implant material. FIG. 10 is a series of photographs illustrating the effect of nitric oxide release material coating an implantable medical device.

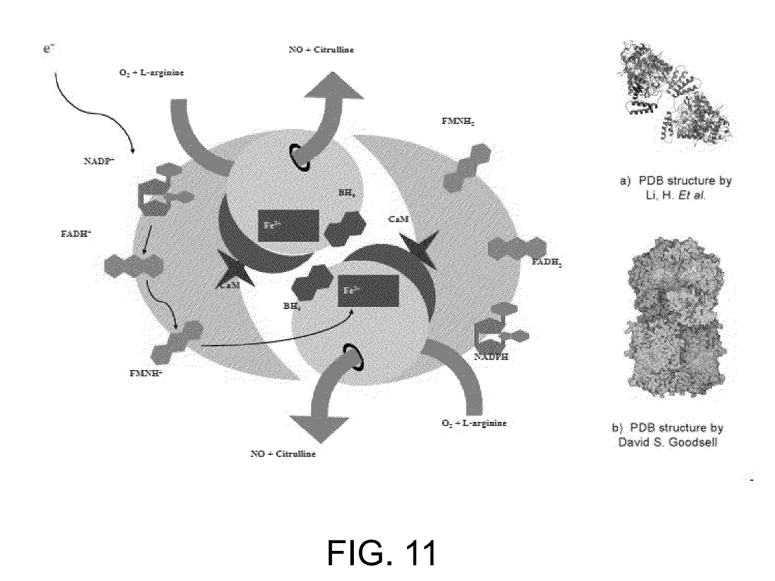

Fiber matrices can be electrospun such that NOS enzymes are encapsulated, embedded, or otherwise included in the electrospun fiber matrices. For example, aqueous pockets formed in the electrospun fibers can include NOS. Such fibers can be a biocompatible platform for nitric oxide release. The substrate molecules and products can flow in and out of the aqueous pockets while the NOS is immobilized by the fiber matrix. FIG. 11 is a schematic representation of NOS domains, two paired enzyme units, and the channeling of an electron with the participation of co-factors, and two PDB structures of NOS.

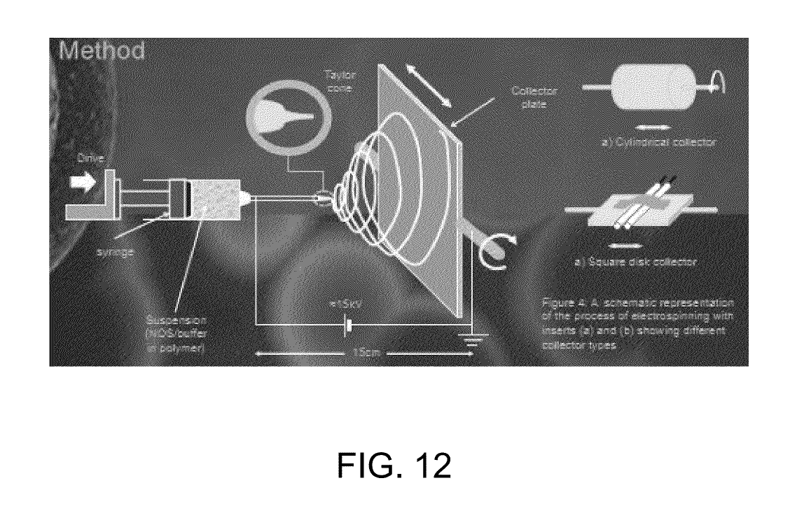

FIG. 12 illustrates a method similar to that illustrated in FIG. 1 for electrospinning fibers for embedding NOS in the electrospun fibers. The polymer used for the solution can be those disclosed herein or other polymers such as polycaprolactone (PCL) and polyurethane (PU). As shown in FIG. 12, a guided stream of polymer solution containing suspended aqueous pockets of enzyme solution can be directed towards a collector drum in a strong electric field. In its path of acceleration towards the target, solvent can evaporate and the charged jet can thin-out leaving a fibrous membrane containing “nodes” of aqueous pockets with encapsulated, entrapped or otherwise included NOS enzymes. As shown in FIG. 12, the target can be a drum or a flat plate configured to rotate.

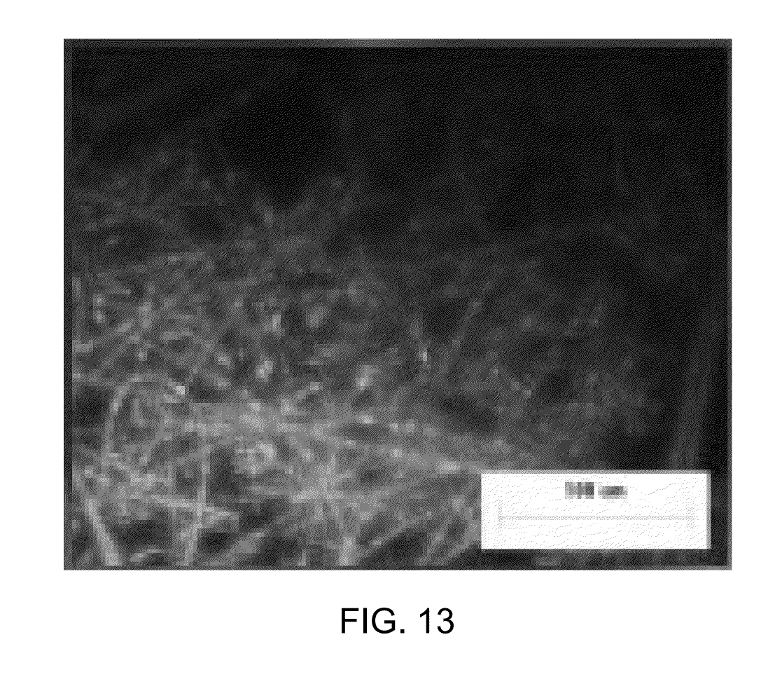

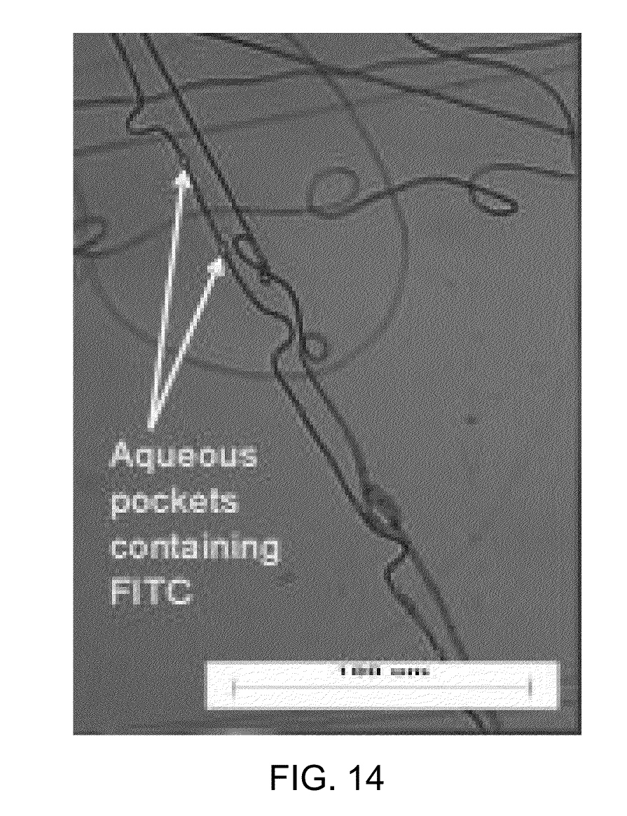

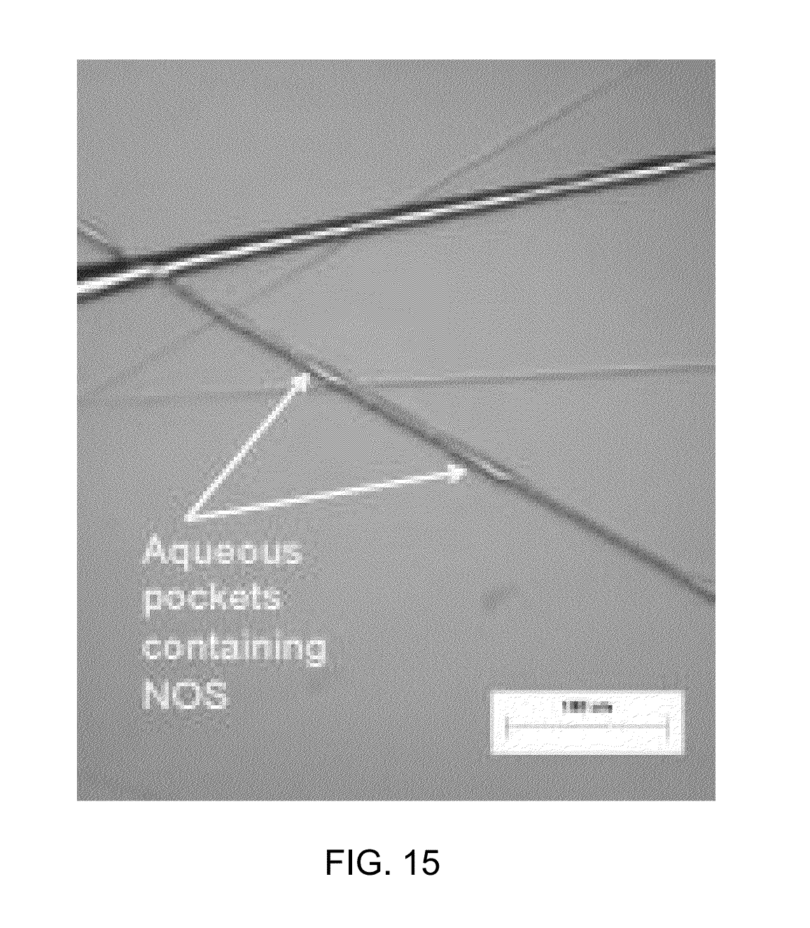

FIGS. 13-15 are magnified images of fibers resulting from the use of differing parameters and conditions. For the fibers of FIG. 13: 1) the spinning mixture was 50:1 10% w/w PCL in CHCl₃:FITC (Fluorescein-5-isothiocyanate) in DI water; 2) the parameters were 15 kV DC voltage, 1.0 ml/hr flow rate, 15 ejection (electrode) distance; and 3) the conditions were 20° C. and 65% humidity. For the fibers of FIG. 14: 1) the spinning mixture was 25:1 10% w/w PCL in CHCl₃:10.0 μM Dextran and FITC; 2) the parameters were 15 kV DC voltage, 1.0 ml/hr flow rate, and 15 ejection (electrode) distance; and 3) the conditions were 20° C. and 67% humidity. For the fibers of FIG. 15: 1) the spinning mixture was 50:1 10% w/w PCL in CHCl₃:NOS in EPPS buffer; 2) the parameters were 15 kV DC voltage, 1.0 ml/hr flow rate, and 15 ejection (electrode) distance; and 3) the conditions were 20° C. and 65% humidity.

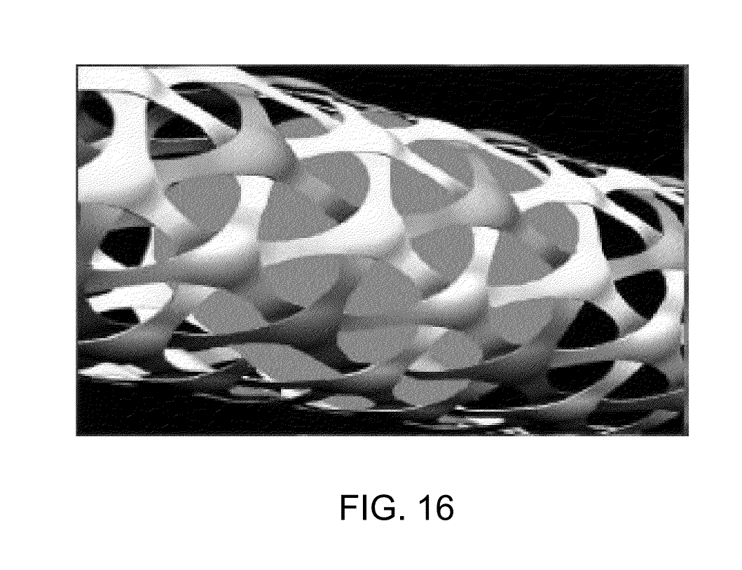

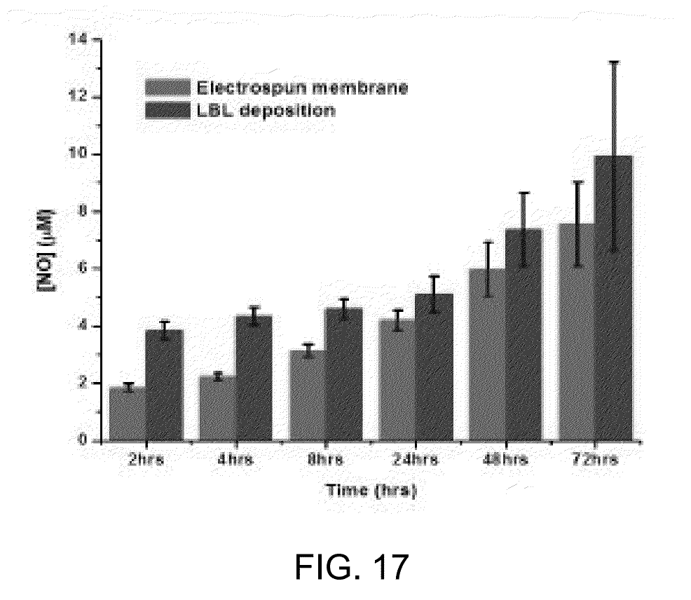

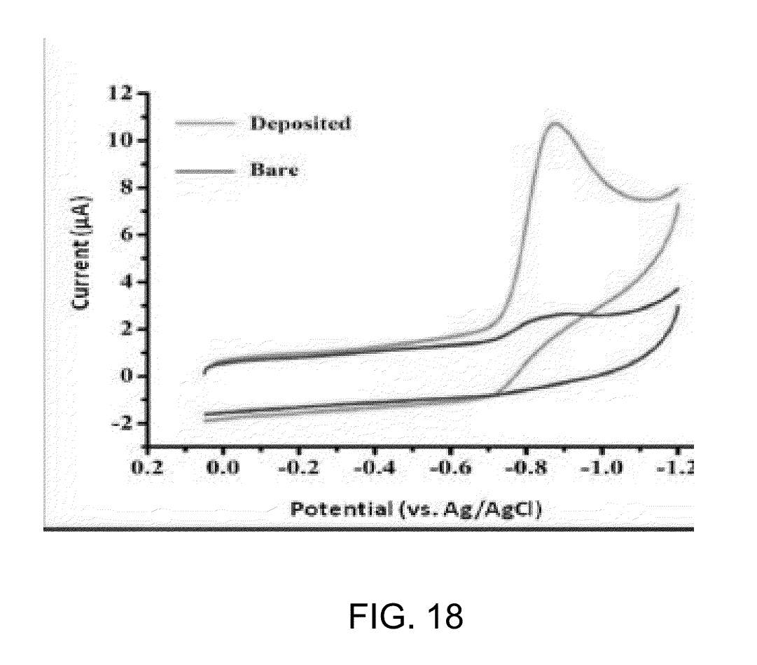

FIG. 16 is a schematic representation of an embedded aqueous “node” inside a fiber matrix. The embedded NOS is protected from degradation by proteases. The substrate molecules and products are able to move through fiber matrix. FIG. 17 is a chart comparing a layer-by-layer method of deposition of NOS on the surface of an implantable biomaterial with the electrospinning method. The chart depicts cumulative nitric oxide concentration in the solution. FIG. 18 depicts the electrochemical characterization of embedded fibers. The chart is a cyclic voltammogram for bare and membrane deposited graphite electrodes at a final nitric oxide concentration of 140 μM.

Embedding or encapsulating NOS in electrospun fiber results in fibers that retain functional integrity, which is due to the aqueous pockets within which the enzyme is entrapped. Electrochemical characterization results are the typical electrocatalytic behavior of the enzyme. The activity of NOS in the electrospun fibers is similar to that of NOS layer-by-layer deposited films. The electrospun fiber allows transport of substrate molecules and products in and out of the pockets and through the polymer matrix.

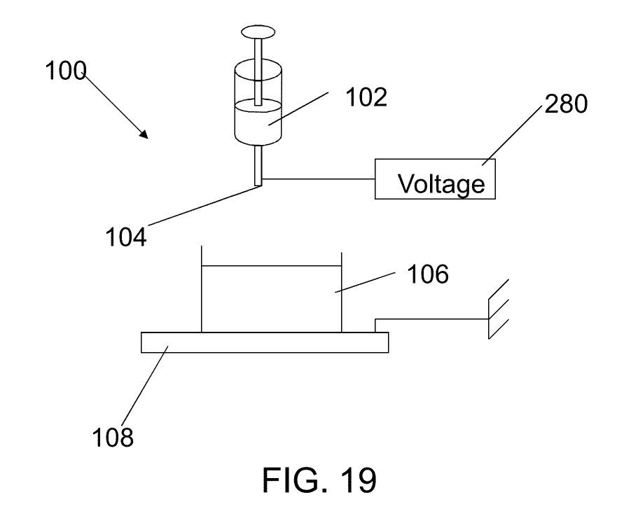

In addition to electrospinning processes, protein dissolved in benign solvents and mixed with agents as described herein can be used to form protein agglomerates such as generally spherical particles or beads. An apparatus 100 for forming protein agglomerate is schematically shown in FIG. 19. Protein agglomerates can be formed using methods that include electrospinning, electrospraying, and gravitational feed methods. The apparatus 100 includes a syringe 102 equipped with a metal needle 104. The syringe 102 is suspended over a receptacle 106, and the receptacle 106 is positioned on a metal plate 108, which is grounded. A protein solution comprising protein and an agent dissolved in a water-alcohol-salt solvent as described herein is placed in the syringe 102. Similar to previous descriptions, an electrical potential can be applied to charge the protein solution by applying a voltage from a power supply 110 to the metal needle 104. A solution of a cross-linking agent such as EDC dissolved in a solvent such as ethanol can be placed in the receptacle 106.

For electrospinning, the electrical potential can be increased to grow the electrostatic forces and overcome the surface tension at a tip of the needle 104. As this surface tension is overcome, a fine jet of protein solution containing entangled protein chains can be drawn out of the needle 104. As the fine jet travels through the air, the solvent evaporates leaving a dry protein fiber that engages the surface of the cross-linking solution in the receptacle 106. The impact of the protein fiber's engagement with the surface of the cross-lining solution fractures the fiber into relatively short sections. Upon entering the cross-linking solutions, each short section of protein fiber draws inward and cross-links with itself, resulting in a generally spherical protein agglomerate or bead. In one example, the protein solvent comprises about 16 percent collagen by weight dissolved in a solvent of about one-to-one ratio by volume of PBS buffer (20×) to ethanol. A flow rate of about 1 ml/h is applied to the protein solution in the syringe 102, a voltage of about 25 kV is applied to the metal needle 104, and the metal plate 108 is spaced about 20 cm from the tip of the metal needle 104. An cross-linking solution of EDC dissolved in ethanol is placed in the receptacle 106. Such protein agglomerates can be, for example, more than 100 micrometers in diameter.

For electrospraying, the tip of the needle 104 and the grounded plate 108 can be placed closer together as compared to the described electrospinning method. Such positioning can result in the protein solution exiting the needle 104 and forming droplets of solution prior to entering the cross-linking solution in the receptacle 106. Such droplets internally cross-link once entering the cross-linking solution and form spherical protein agglomerates or beads. Such protein agglomerates can be, for example, approximately 2 to 3 micrometers in diameter.

For a gravitation feed method, no electrical potential is needed. Gravity is used to draw beads of protein solution from the needle 104. The beads fall into the cross-linking solution and internally cross-link forming generally spherical protein agglomerates. Alternatively, each bead can break up into smaller beads upon impact with the surface of the cross-linking solution. Such protein agglomerates can be, for example, approximately 20 to 30 micrometers in diameter.

Parameters such as the distance between the tip of the needle 104 and the metal plate 108, flow rate of protein solution from the needle 104, voltage applied to the needle 104, concentration of protein in the protein solution, concentration of salt in the protein solution, and ratio of alcohol to water in the protein solution can affect the size of protein agglomerates or beads. However, for comparatively similar parameters, electrospraying can produce the smallest protein agglomerates, gravity feed can produce protein agglomerates larger than electrospraying, and electrospinning can produce protein agglomerates larger than the gravitational feed method.

Protein dissolved in benign solvents as described herein can be used to form porous protein films, scaffolds and gels. In one example, a protein solution can be deposited in a receptacle so that the protein solution covers the bottom of the receptacle. A solution that includes a cross-linking agent such as EDC in ethanol is poured over the protein solution. In one example, the cross-linking solution comprises about 0.2 millimoles of EDC. The receptacle can be hermetically covered for a period of time, for example about 24 hours. Evaporation of the solution results in a protein film forming on the bottom of the receptacle. Some salt crystals may be present on the surface of the film. Such salt crystals can be removed by washing the film with deionized water, which can leach out the salt. Once the salt is leached out, the film is left with a porous structure that includes numerous pores that intersect forming a protein structure with an open network of pores. The porous structure of the film can include pores that range from submicron in size to over 30 micrometers in size.

Such a film with intersection pores can be suitable as a scaffold for cell repopulation or tissue growth. It will be understood that the intersecting pores mimic the ECM structure of human tissue and provide expanded surfaces on which cells and tissue can grow.

An example of another method of forming a protein structure with an open network of pores is hereafter described. A protein solution as described herein is prepared in a receptacle and stirred. As the protein solution is stirred, a cross-linking solution including a cross-linking agent such as EDC is deposited in the receptacle. Stirring continues until a protein cross-links and forms a gel in the receptacle. Once cross-linked the protein gel can be rinsed with deionized water to remove salts and alcohol from the gel. The gel is quenched in liquid nitrogen and frozen. The gel is placed in a vacuum chamber and water in or on the gel sublimes or otherwise evaporates. Such a method results in a low density protein scaffold with foam-like properties and an open network of pores. The pores as shown range in size from about 10 micrometers to about 50 micrometers. The size of the pores can be controlled by varying the protein content in the protein solution and the buffer to alcohol ratios.

The foregoing description of examples has been presented for purposes of illustration and description. It is not intended to be exhaustive or limiting to the forms described. Numerous modifications are possible in light of the above teachings. Some of those modifications have been discussed, and others will be understood by those skilled in the art. The examples were chosen and described in order to best illustrate principles of various examples as are suited to particular uses contemplated. The scope is, of course, not limited to the examples set forth herein, but can be employed in any number of applications and equivalent devices by those of ordinary skill in the art.

What is claimed is:

1. A fiber encapsulating an agent substantially as shown and described herein.

2. A fiber encapsulating an agent according to one or more of the inventive principles as shown and described herein.

3. A method of forming a fiber encapsulating an agent, the method being substantially as shown and described herein.

4. A method of forming a fiber encapsulating an agent, the method according to one or more of the inventive principles as shown and described herein.

5. A method of using a fiber encapsulating an agent, the method being substantially as shown and described herein.

6. A method of using a fiber encapsulating an agent, the method according to one or more of the inventive principles as shown and described herein.

7. A fiber with an agent embedded in said fiber substantially as shown and described herein.

8. A fiber with an agent embedded in said fiber according to one or more of the inventive principles as shown and described herein.

9. A method of forming a fiber with an agent embedded in said fiber, the method being substantially as shown and described herein.

10. A method of forming a fiber with an agent embedded in said fiber, the method according to one or more of the inventive principles as shown and described herein.

11. A method of using a fiber with an agent embedded in said fiber, the method being substantially as shown and described herein.

12. A method of using a fiber with an agent embedded in said fiber, the method according to one or more of the inventive principles as shown and described herein.
